(19) United States
(12) Patent Application Publication
(10) Pub. No.: US 2025/0104860 A1
(43) Pub. Date: Mar. 27, 2025

(54) SYSTEMS AND METHODS FOR REAL-TIME DETERMINATIONS OF MENTAL HEALTH DISORDERS USING MULTI-TIER MACHINE LEARNING MODELS BASED ON USER INTERACTIONS WITH COMPUTER SYSTEMS

(71) Applicant: OPTIMUM HEALTH LTD, London (GB)

(72) Inventors: Helen YANNAKOUDAKIS, London (GB); Erika BRODNOCK, London (GB)

(73) Assignee: OPTIMUM HEALTH LTD, London (GB)

(21) Appl. No.: 18/502,732

(22) PCT Filed: May 6, 2021
(86) PCT No.: PCT/EP2021/062085

§ 371 Date: Nov. 6, 2023

(51) Int. Cl. G16H 50/20; G10L 15/16; G10L 15/197; G16H 20/70

(52) U.S. Cl. CPC G16H 50/20; G10L 15/16; G10L 15/197; G16H 20/70

(57) ABSTRACT

The methods and systems use a novel machine learning architecture, specifically a multi-tier architecture in which each tier functions to produce specific outputs that contribute to an overall diagnosis of mental health disorders based on user interactions. For example, the first tier of the machine learning model is trained to understand the words, contexts, and meanings of user inputs into a computer system. A second tier of the machine learning model is trained to provide real-time determinations of mental health disorders indirectly through the use of emotional states.

### BACKGROUND

In recent years, the number of diagnoses for, and variety of, mental health disorders has been growing. Unfortunately, unlike many physical health disorders, there is a dearth of biological markers that can be used to detect or track a decline in mental health or wellbeing. Conventional techniques therefore rely heavily on users' self-reporting and describing of their symptoms. This commonly requires users to identify a change in their behaviour and seek help, which often occurs only after a significant decline in wellbeing. Furthermore, the large time between a users' desire to seek help and their ability to meet with someone for professional help, means that the description of symptoms is dependent on memory, which is often biased by a current mental state. Accordingly, there is a need to identify mental health disorders in a real-time and automated way.

### SUMMARY

Methods and systems are described herein for real-time determination of mental health disorders using multi-tier machine learning models based on user interactions with computer systems. For example, machine learning techniques described herein used in natural language processing show to identify and contextualize user inputs (e.g., whether based on textual, audio, and/or video inputs). Unfortunately, self-identifying and describing mental health symptoms are difficult for users to describe in consistent and categorical ways, limiting the benefit of conventional machine learning models that rely on natural language processing.

However, as described herein, the methods and systems use a novel machine learning architecture, specifically a multi-tier architecture in which each tier functions to produce specific outputs that contribute to an overall diagnosis of mental health disorders based on user interactions. For example, the first tier of the machine learning model is trained to understand the words, contexts, and meanings of user inputs into a computer system. Additionally, first tier analyses current interactions (e.g., text data) whenever it receives inputs from the user and may therefore track individuals in a real-time manner that is free from recall bias.

In view of this architecture, however, another technical challenge arises. For example, even if the first tier of the system is be trained to understand users, users may not know how to describe their symptoms, may not be aware of their conditions, or may not be aware of mental health disorders in general. Furthermore, each user is unique and there is no one-to-one mapping between a given symptom or user's feeling and a diagnosis of a particular mental health disorder. Recognizing this fluidity and lack of a standardized taxonomy, the second tier of the system is trained to provide real-time determinations of mental health disorders indirectly through the use of emotional states. For example, the second tier, in contrast to the first tier, is trained to generate specific recommendations as to mental health disorders based on emotional states using the words, contexts, and meanings determined by the first tier. In particular, the system operates to provide language processing algorithms that may annotate inputs with emotion labels and provide recommendations on mental health disorders.

For example, mental health disorders involve changes (or lack thereof) in emotions experienced. For example, depression is typically characterised by feelings of sadness and loss of motivation. Additionally, users are more likely to be able to identify how they feel emotionally as opposed to describe their potential mental health disorder. Moreover, in cases where users still have difficulty expressing themselves, describing their emotions provides a level of fluidity that does not foreclose a given diagnosis based on any outlying emotions and/or abnormal description of emotion. This however creates a novel problem in that the computer system is required to interpret users' descriptions of emotional states, which may also be unreliable.

To overcome this novel technical problem, the system may generate a specialized taxonomy of emotional descriptions, and uses machine learning algorithms that may generate and interpret multivariate, and potentially conflicting, factors. The machine learning models may generate outputs in a vectorized form that may then be used to provide a recommendation of a mental health disorder based on a determined emotional state. For example, using the vectorized form, the methods and systems may combine emotional diagnosis that may be generated using parallel systems that use different, and potentially contradictory, standards. The nuance and flexibility of language and user descriptions, which may only implicitly indicate an emotional state, is captured as a probability score that is analysed to generate a recommended mental health disorder.

For example, the system may link mental health to the emotional state of the speaker and may detect both through affective characteristics of their language. For example, by detecting changes in the user's use of words as well as the linguistic characteristics and absolutism of their language, the system may detect cues to one's mental health. As such, the system may specialize its natural language processing capability (e.g., through the multi-tier structure) capture the state of a user and make predictions without asking users to explicitly reflect on their mental health.

In some aspects, the methods and systems are disclosed for generating mental health disorder recommendations using multi-tier machine learning models. For example, the system may receive a first user action during a conversational interaction with a user interface. The system may determine a first feature input based on the first user action in response to receiving the first user action. The system may input the first feature input into a first machine learning model, wherein the first machine learning model is trained to select a context from a plurality of contexts based on the first feature input, and wherein each context of the plurality of contexts corresponds to a respective emotional state of a user. The system may receive a first output from the first machine learning model. The system may input the first output into a second machine learning model, wherein the second machine learning model is trained to select an emotional state from a plurality of emotional states of the selected context based on the first output, and wherein each emotional state of the plurality of emotional states corresponds to a respective emotional state of the user. The system may receive a second output from the second machine learning model. The system may select a mental health disorder recommendation from a plurality of mental health disorder recommendations based on the second output. The system may generate, at the user interface, the mental health disorder recommendation following the conversational interaction.

Various other aspects, features, and advantages of the invention will be apparent through the detailed description of the invention and the drawings attached hereto. It is also to be understood that both the foregoing general description and the following detailed description are examples and not restrictive of the scope of the invention. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification “a portion,” refers to a part of, or the entirety of (i.e., the entire portion), a given item (e.g., data) unless the context clearly dictates otherwise.

### BRIEF DESCRIPTION OF THE DRAWINGS

FIG. 1 shows an illustrative user interface for receiving information upon which to generate recommendations using multi-tier machine learning models, in accordance with one or more embodiments.

FIG. 2 is an illustrative system for generating mental health disorder recommendations using multi-tier machine learning models, in accordance with one or more embodiments.

FIG. 3A is an illustrative diagram of model predicted labels, in accordance with one or more embodiments.

FIG. 3B is an illustrative diagram of n-grams used by a machine learning model of the multi-tier machine learning models, in accordance with one or more embodiments.

FIG. 3C is an illustrative diagram of emotional state accuracy scores generated by a machine learning model of the multi-tier machine learning models, in accordance with one or more embodiments.

3D is an illustrative diagram of emotional state F1 scores generated by a machine learning model of the multi-tier machine learning models, in accordance with one or more embodiments.

FIG. 4 is an illustrative model architecture of a multi-tier machine learning model, in accordance with one or more embodiments.

FIG. 5 shows a flowchart of the steps involved in generating mental health disorder recommendations using multi-tier machine learning models, in accordance with one or more embodiments.

FIG. 6 shows a flowchart of the steps involved in generating mental health disorder recommendations using a two-tier machine learning model, in accordance with one or more embodiments.

### DETAILED DESCRIPTION OF THE DRAWINGS

In the following description, for the purposes of explanation, numerous specific details are set forth in order to provide a thorough understanding of the embodiments of the invention. It will be appreciated, however, by those having skill in the art, that the embodiments of the invention may be practiced without these specific details or with an equivalent arrangement. In other cases, well-known structures and devices are shown in block diagram form in order to avoid unnecessarily obscuring the embodiments of the invention.

The methods and systems use a novel machine learning architecture, specifically a multi-tier architecture in which each tier functions to produce specific outputs that contribute to an overall diagnosis of mental health disorders based on user interactions. For example, the system may predict the affectual states of the users as a useful inductive bias for predicting mental ill-health. The system may additionally and/or alternatively simultaneously predict emotion, sentiment, and/or mental health (e.g., via multi-task machine learning) using multi-modal data (i.e., text, audio, video, etc.) to capture a diverse set of features that, in turn, are used by the system to generate more accurate predictions and better generalisation, as well as help us move towards building personalised/adaptive models (as different users may experience the same emotion in different ways).

For example, the multi-tier machine learning models may use a language processing algorithm that may annotate input text with emotion labels. For example, the system may use Paul Ekman's model of emotions which involves happiness, sadness, anger, fear, surprise, and disgust. In contrast, the dimensional framework views emotional expression as being interrelated through variations on a small number of independent dimensions. The model in this framework describes emotions with a score on each of 3 dimensions: valence (positivity/negativity), arousal (excitedness/apathy), dominance (degree of control).

The multi-tier machine learning models may use supervised and/or unsupervised machine learning method to develop models that are able to label text with emotions using both frameworks. Supervised learning requires a pre-labelled dataset such that text examples are input into the multi-tier machine learning model, which may then output emotion predictions. The multi-tier machine learning model uses the pre-labelled dataset during training as it learns to minimise the error between its predictions and the true labels.

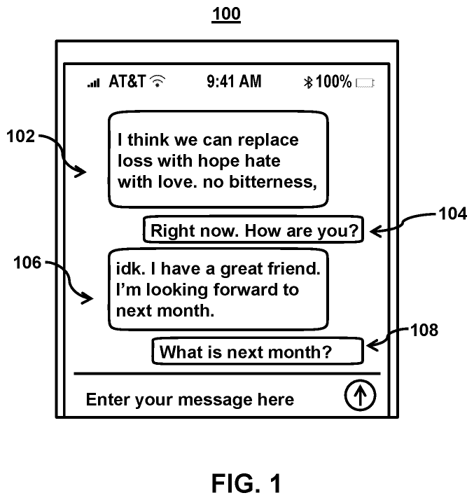

FIG. 1 shows an illustrative user interface for receiving information upon which to generate recommendations using multi-tier machine learning models, in accordance with one or more embodiments. For example, FIG. 1 shows user interface 100. The system (e.g., a mobile application) may generate and respond to user interactions in a user interface (e.g., user interface 100) in order to engage in a conversational interaction with the user. The conversational interaction may include a back-and-forth exchange of ideas and information between the system and the user. The conversational interaction may proceed through one or more mediums (e.g., text, video, audio, etc.).

In order to maintain the conversational interaction, the system may need to generate recommendations (e.g., a mental health disorder recommendation and/or a recommendation for behaviour modification) dynamically and/or in substantially real-time. For example, the system may generate mental health disorder recommendations. In some embodiments, the system may continually determine a likely emotional state of the user in order to generate mental health disorder recommendations (e.g., in the form of prompts, notifications, and/or other communications) to the user and/or other users (e.g., a mental health professional). It should be noted that a mental health disorder recommendation may be included with any action (or inaction) taken by the system, including computer processes, which may or may not be perceivable to a user.

For example, in response to a user action, which in some embodiments may comprise a user, inputting a text input (e.g., user input 102) into user interface 100, logging into an application, and/or a prior action (or lack thereof) by a user to a prior response generated by the system, the system may take one or more steps to generate mental health disorder recommendations. These steps may include retrieving data about the user, retrieving data from other sources, monitoring user actions, and/or other steps in order to generate a feature input (e.g., as discussed below).

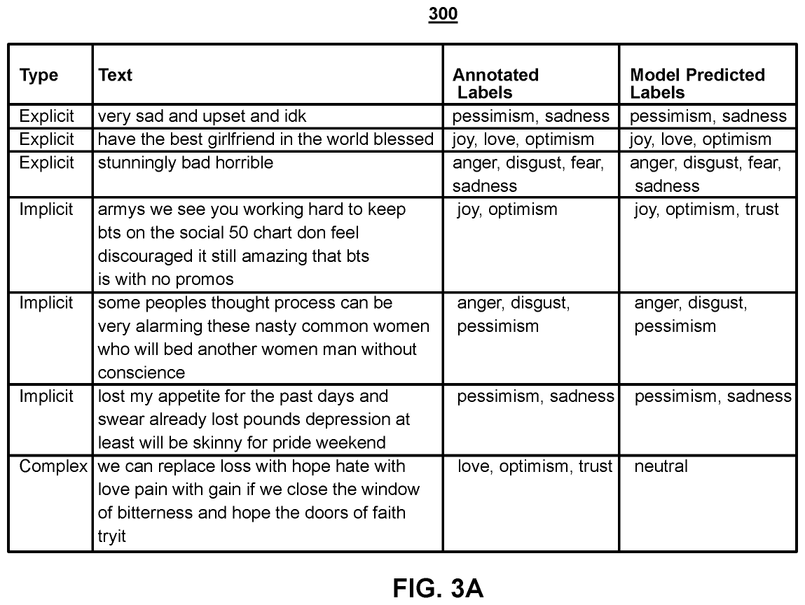

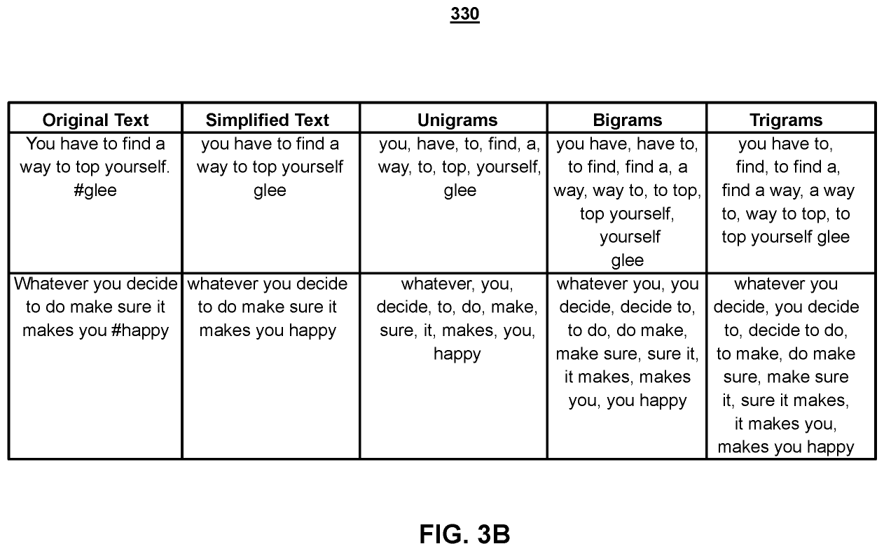

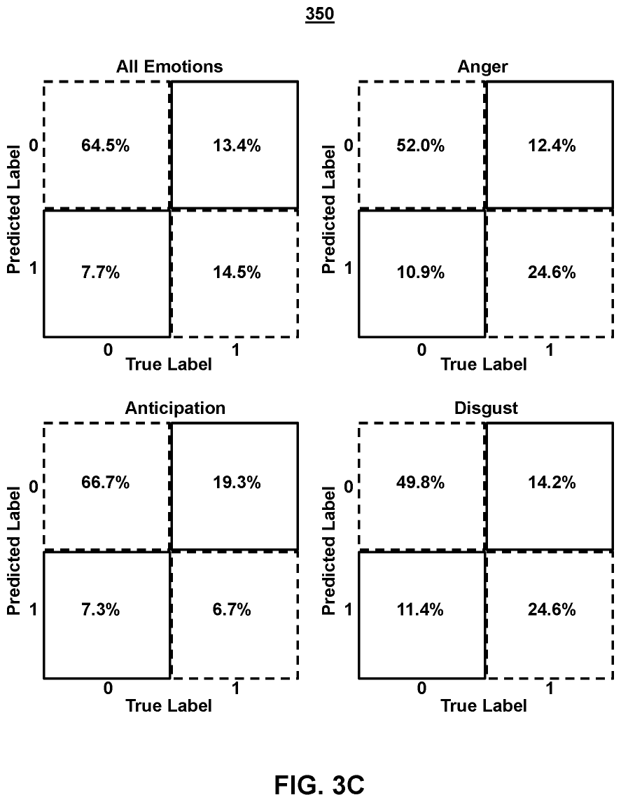

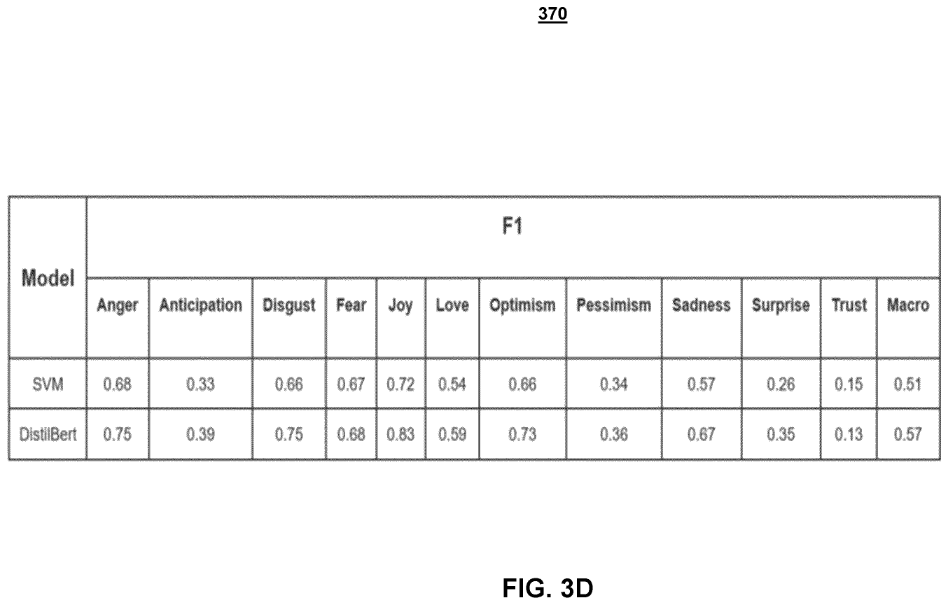

The system may also generate additional queries (e.g., query 104 and query 108) to a user to receive additional information about a user (e.g., for use in determining a mental health disorder recommendation). Additionally or alternatively, the system may receive additional user actions (e.g., second user input 106) that the system may use to generate mental health disorder recommendations. For example, in some embodiments, emotional states, which may be used to generate mental health disorder recommendations, may be explicitly described. Alternatively or additionally, the emotional states may be complex and/or implicit (e.g., as further described in FIG. 3A below). Accordingly, the system may issue multiple queries where the text and/or subject matter of the query is aimed at causing a user to generate a user input that aids the system in predicting an emotional state. Additionally or alternatively, the system may generate multiple queries if the system's confidence in a determination does not meet a threshold confidence (e.g., as reflected by a probability distribution over different sets of emotions).

The system may generate a feature input based on the user action or actions. The system may generate the feature input by creating vector representations of this information. For example, the system may take characteristics of user actions represented as mathematical vectors as inputs and output emotion labels. The system may train the model to best categorise the input vectors with emotions labels involves optimising the position of a linear plane to best-partition. In some embodiments, the system may use linear and/or non-linear models.

In some embodiments, upon determination of a mental health disorder recommendation, the system may transmit a mental health disorder recommendation to another user (e.g., a mental health professional). For example, the system may compare the mental health disorder recommendation to a list of mental health disorder recommendations that requires treatment. In response to detecting a match, the system may transmit the mental health disorder recommendation to the other user (e.g., at another user interface). The user may then contact (e.g., via the same or a different platform) the other user, or the other user may receive contact information with the mental health disorder recommendation. The other user may be a mental health professional associated with a provider of the application or mental health services. In order to improve the interaction between the first and second user, the system may provide recommendations to the second user about potential questions, symptoms, disorders, etc. that the first user may have. Additionally or alternatively, the system may access location information in the system of the second user, determine a network pathway to quickly and efficiently retrieve this information, and/or pre-fetch this information. In each case, the system may more efficiently (and in less time) obtain relevant information that may inform the second user about potential questions, symptoms, disorders, etc. of the first user.

For example, the system may generate, at a user interface corresponding to a second user, the mental health disorder recommendation for a first user. For example, the system may, in response to detecting a user action, monitor for another user action, wherein the other user action is not received via the user interface, and wherein the other user action corresponds to a first user contacting a second user. In response to detecting the other user action, the system may generate, at a second user interface corresponding to the second user, the mental health disorder recommendation selected by the system.

The system may then determine user information (e.g, medical history, symptoms, treatment options, etc.) corresponding to the mental health disorder recommendation. For example, the system may determine user information corresponding to the mental health disorder recommendation generated by the user action at the first user interface. Additionally or alternatively, the system may determine a network location of the user information. For example, the system may determine a network location of the user information (e.g., on a network associated with the second user interface). The network location may correspond to a computer domain and/or file featuring information related to the mental health disorder recommendation and/or a computer domain or file corresponding to the user information (e.g., medical history, symptoms, treatment options, etc.)

The system may then generate a network pathway to the user information. For example, the system may generate a network pathway (e.g., on the network associated with the second user interface) to the user information. The system may then automatically retrieve the user information from the network location. For example, the system may automatically retrieve the user information from the network location in response to the other user action. The system may then generate for display the user information on the second user interface. For example, the system may generate for display the user information on the second user interface of a second user, wherein the second user interface is located remotely from the first user interface.

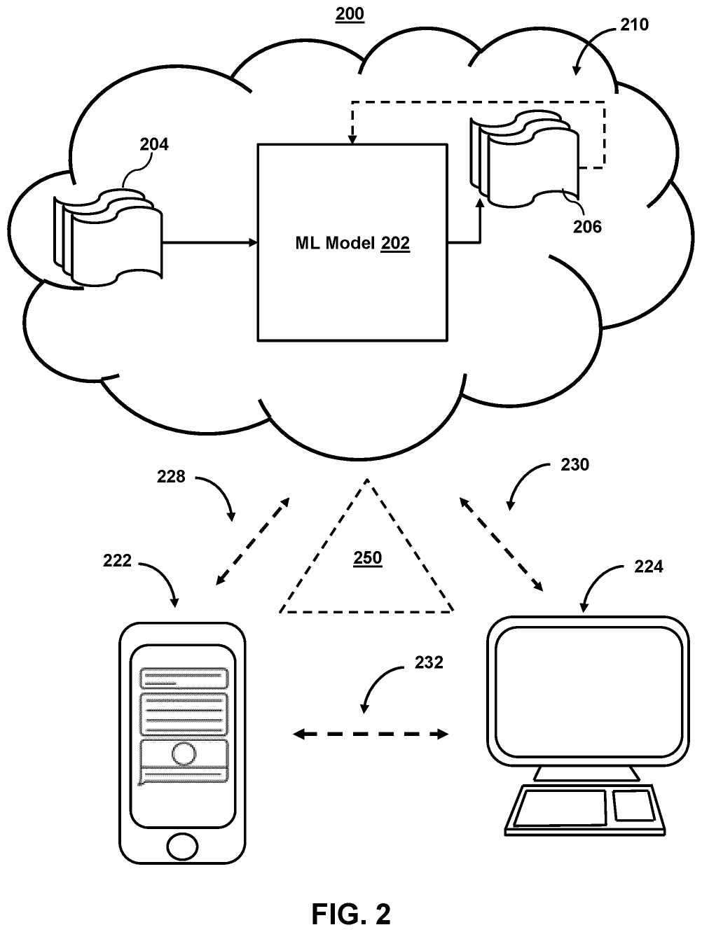

FIG. 2 shows an illustrative system for generating mental health disorder recommendations using multi-tier machine learning models. For example, system 200 may represent the components used for generating mental health disorder recommendations as shown in FIG. 1. As shown in FIG. 2, system 200 may include mobile device 222 and user terminal 224. While shown as a smartphone and personal computer, respectively, in FIG. 2, it should be noted that mobile device 222 and user terminal 224 may be any computing device, including, but not limited to, a laptop computer, a tablet computer, a hand-held computer, other computer equipment (e.g., a server), including “smart,” wireless, wearable, and/or mobile devices. FIG. 2 also includes cloud components 210. Cloud components 210 may alternatively be any computing device as described above and may include any type of mobile terminal, fixed terminal, or other device. For example, cloud components 210 may be implemented as a cloud computing system and may feature one or more component devices. It should also be noted that system 200 is not limited to these three devices. Users may, for instance, utilize one or more other devices to interact with one another, one or more servers, or other components of system 200. It should be noted that, while one or more operations are described herein as being performed by particular components of system 200, those operations may, in some embodiments, be performed by other components of system 200. As an example, while one or more operations are described herein as being performed by components of mobile device 222, those operations may, in some embodiments, be performed by components of cloud components 210. In some embodiments, the various computers and systems described herein may include one or more computing devices that are programmed to perform the described functions. Additionally or alternatively, multiple users may interact with system 200 and/or one or more components of system 200. For example, in one embodiment, a first user and a second user may interact with system 200 using two different components.

With respect to the components of mobile device 222, user terminal 224, and cloud components 210, each of these devices may receive content and data via input/output (hereinafter “I/O”) paths. Each of these devices may also include processors and/or control circuitry to send and receive commands, requests, and other suitable data using the I/O paths. The control circuitry may comprise any suitable processing, storage, and/or input/output circuitry. Each of these devices may also include a user input interface and/or user output interface (e.g., a display) for use in receiving and displaying data. For example, as shown in FIG. 2, both mobile device 222 and user terminal 224 include a display upon which to display data (e.g., based on recommended contact strategies).

Additionally, as mobile device 222 and user terminal 224 are shown as touchscreen smartphones, these displays also act as user input interfaces. It should be noted that in some embodiments, the devices may have neither user input interface nor displays and may instead receive and display content using another device (e.g., a dedicated display device such as a computer screen and/or a dedicated input device such as a remote control, mouse, voice input, etc.). Additionally, the devices in system 200 may run an application (or another suitable program). The application may cause the processors and/or control circuitry to perform operations related to generating mental health disorder recommendations using multi-tier machine learning models.

Each of these devices may also include electronic storages. The electronic storages may include non-transitory storage media that electronically stores information. The electronic storage media of the electronic storages may include one or both of (i) system storage that is provided integrally (e.g, substantially non-removable) with servers or client devices, or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information obtained from servers, information obtained from client devices, or other information that enables the functionality as described herein.

FIG. 2 also includes communication paths 228, 230, and 232. Communication paths 228, 230, and 232 may include the Internet, a mobile phone network, a mobile voice or data network (e.g., a 4G or LTE network), a cable network, a public switched telephone network, or other types of communications networks or combinations of communications networks. Communication paths 228, 230, and 232 may separately or together include one or more communications paths, such as a satellite path, a fibre-optic path, a cable path, a path that supports Internet communications (e.g., IPTV), free-space connections (e.g., for broadcast or other wireless signals), or any other suitable wired or wireless communications path or combination of such paths. The computing devices may include additional communication paths linking a plurality of hardware, software, and/or firmware components operating together. For example, the computing devices may be implemented by a cloud of computing platforms operating together as the computing devices.

Cloud components 210 may be a database configured to store user data for a user. For example, the database may include user data that the system has collected about the user through prior transactions. Alternatively, or additionally, the system may act as a clearing house for multiple sources of information about the user. Cloud components 210 may also include control circuitry configured to perform the various operations needed to generate recommendations. For example, the cloud components 210 may include cloud-based storage circuitry configured to store a first machine learning model and a second machine learning model. Cloud components 210 may also include cloud-based control circuitry configured to determine an emotional state of the user based on a multi-tier machine learning model. Cloud components 210 may also include cloud-based input/output circuitry configured to generate the mental health disorder recommendation following the conversational interaction.

Cloud components 210 may include machine learning model 202. Machine learning model 202 may take inputs 204 and provide outputs 206. The inputs may include multiple datasets such as a training dataset and a test dataset. Each of the plurality of datasets (e.g., inputs 204) may include data subsets related to user data, contact strategies, and results. In some embodiments, outputs 206 may be fed back to machine learning model 202 as input to train machine learning model 202 (e.g., alone or in conjunction with user indications of the accuracy of outputs 206, labels associated with the inputs, or with other reference feedback information) In another embodiment, machine learning model 202 may update its configurations (e.g., weights, biases, or other parameters) based on the assessment of its prediction (e.g., outputs 206) and reference feedback information (e.g., user indication of accuracy, reference labels, or other information). In another embodiment, where machine learning model 202 is a neural network, connection weights may be adjusted to reconcile differences between the neural network's prediction and the reference feedback. In a further use case, one or more neurons (or nodes) of the neural network may require that their respective errors are sent backward through the neural network to facilitate the update process (e.g., backpropagation of error). Updates to the connection weights may, for example, be reflective of the magnitude of error propagated backward after a forward pass has been completed. In this way, for example, the machine learning model 202 may be trained to generate better predictions.

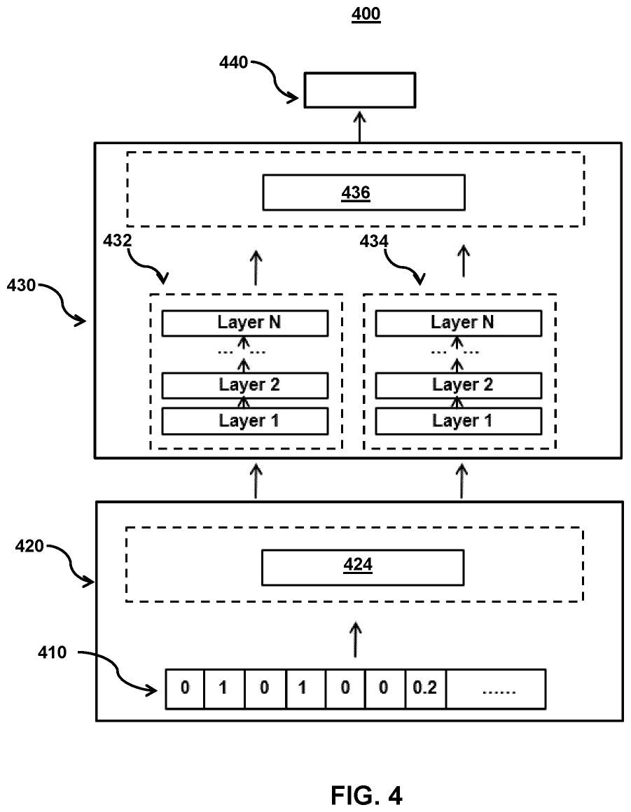

In some embodiments, machine learning model 202 may include an artificial neural network (e.g., as described in FIG. 4 below). In such embodiments, machine learning model 202 may include an input layer and one or more hidden layers. Each neural unit of machine learning model 202 may be connected with many other neural units of machine learning model 202. Such connections can be enforcing or inhibitory in their effect on the activation state of connected neural units. In some embodiments, each individual neural unit may have a summation function which combines the values of all of its inputs together. In some embodiments, each connection (or the neural unit itself) may have a threshold function such that the signal must surpass before it propagates to other neural units. Machine learning model 202 may be self-learning and trained, rather than explicitly programmed, and can perform significantly better in certain areas of problem solving, as compared to traditional computer programs. During training, an output layer of machine learning model 202 may correspond to a classification of machine learning model 202 and an input known to correspond to that classification may be input into an input layer of machine learning model 202 during training. During testing, an input without a known classification may be input into the input layer, and a determined classification may be output.

For example, in one model (e.g., based on 11 emotional states and/or the absence of one or more of the emotional states), the system may use emotional states that include anger (also includes annoyance, rage); anticipation (also includes interest, vigilance); disgust (also includes disinterest, dislike, loathing); fear (also includes apprehension, anxiety, terror); joy (also includes serenity, ecstasy): love (also includes affection); optimism (also includes hopefulness, confidence). pessimism (also includes cynicism, no confidence); sadness (also includes pensiveness, grief); surprise (also includes distraction, amazement); and trust (also includes acceptance, liking, admiration).

The absence of all 11 emotions may also be used by the system to define an example as neutral/no emotion (e.g., the lack of emotion/interest and apathy also being a key indicator of mental wellbeing). It should be noted that whilst the positive and negative emotions will tend to cluster separately, there is added utility in labelling texts with a broader range of specific emotions (compared to simply positive and negative affect) as these may lead to distinct actions and advice. Unlike many sentiment-labelled datasets, this dataset allowed for each text example to be labelled with multiple emotions (rather than a single emotion label per text example), which better represents human expression through natural language.

In some embodiments, machine learning model 202 may include multiple layers (e.g., where a signal path traverses from front layers to back layers). In some embodiments, back propagation techniques may be utilized by machine learning model 202 where forward stimulation is used to reset weights on the “front” neural units. In some embodiments, stimulation and inhibition for machine learning model 202 may be more free-flowing, with connections interacting in a more chaotic and complex fashion. During testing, an output layer of machine learning model 202 may indicate whether or not a given input corresponds to a classification of machine learning model 202 (e.g., whether context corresponds to a given emotional state and/or whether the given emotional state corresponds to a particular mental health disorder).

System 200 also includes API layer 250. In some embodiments, API layer 250 may be implemented on mobile device 222 or user terminal 224. Alternatively or additionally, API layer 250 may reside on one or more of cloud components 210. API layer 250 (which may be a REST or Web services API layer) may provide a decoupled interface to data and/or functionality of one or more applications. API layer 250 may provide a common, language-agnostic way of interacting with an application. Web services APIs offer a well-defined contract, called WSDL, that describes the services in terms of its operations and the data types used to exchange information. REST APIs do not typically have this contract; instead, they are documented with client libraries for most common languages including Ruby, Java, PHP, and JavaScript. SOAP Web services have traditionally been adopted in the enterprise for publishing internal services, as well as for exchanging information with partners in B2B transactions.

API layer 250 may use various architectural arrangements. For example, system 200 may be partially based on API layer 250, such that there is strong adoption of SOAP and RESTful Web-services, using resources like Service Repository and Developer Portal, but with low governance, standardization, and separation of concerns. Alternatively, system 200 may be fully based on API layer 250, such that separation of concerns between layers like API layer 250, services, and applications are in place.

In some embodiments, the system architecture may use a microservice approach. Such systems may use two types of layers: a Front-End Layer and a Back-End Layer, where microservices reside in this kind of architecture. The role of the API layer 250 in some cases is to provide integration between Front-End and Back-End. In such cases, API layer 250 may use RESTful APIs (exposition to front-end or even communication between microservices). API layer 250 may use AMQP (e.g., Kafka, RabbitMQ, etc.). API layer 250 may use incipient usage of new communications protocols, such as gRPC, Thrift, etc.

In some embodiments, the system architecture may use an open API approach. In such cases, API layer 250 may use commercial or open source API Platforms and their modules. API layer 250 may use a developer portal. API layer 250 may use strong security constraints applying WAF and DDOS protection, and API layer 250 may use RESTful APIs as standard for external integration.

FIGS. 3A-C illustrative diagrams of features and model predicted labels, in accordance with one or more embodiments. In some embodiments, due to the complexity inherent in written text, audio, and/or video inputs, the system may simplify a user input. For example, for a text input, the system may remove punctuation.

FIG. 3A is an illustrative diagram of model predicted labels, in accordance with one or more embodiments. For example, the system may generate queries (e.g., query 104 and query 106 (FIG. 1)) to a user to receive additional information about a user (e.g., for use in determining a mental health disorder recommendation). For example, in some embodiments, emotional states, which may be used to generate mental health disorder recommendations, may be explicitly described. Alternatively or additionally, the emotional states may be complex and/or implicit (e.g., as further described in FIG. 3A below). Accordingly, the system may issue multiple queries where the text and/or subject matter of the query is aimed at causing a user to generate a user input that aids the system in predicting an emotional state.

For example, the system may receive user inputs that include words or phrases associated with a given emotional state. The system may also receive longer text examples that may be made up of both positive and negative emotion words (e.g., user input 102 (FIG. 1), and only some of these words and/or phrases may be relevant to the overall sentiment of the phrase. In these cases, the system may require additional information to understand the context in which the words are being used. For example, the text in user input 102 (FIG. 1) that the model classifies as no emotion/neutral, despite the true labels being love, optimism and trust. To better predict the emotional state, the system may generate query 104 (FIG. 1) that elicits an additional user input (e.g., user input 106 (FIG. 1)). Additionally or alternatively, the system may record and/or use a history of the user into account (e.g., history of conversations and how their affectual state, and the model predictions, change over time).

FIG. 3B is an illustrative diagram of n-grams used by a machine learning model of the multi-tier machine learning models, in accordance with one or more embodiments. For example, diagram 330 illustrates examples of text inputs from the training dataset, and the resulting unigrams, bigrams and trigrams. For example, the total unique features are extracted from the training data and used to build a vocabulary.

For example, the system may train the model to learn the association of words in input texts and the corresponding emotion labels. For example, if single words are used, this is referred to as word unigrams, however using two or three consecutive words can also be used as features, known as bigrams and trigrams. The potential advantage of bigrams and trigrams is that they add more information about the context in which a word is being used, which can be very useful in the English language, where the same word can have different meanings. However, the cost of this is that it can increase feature sparsity as the number of times a unigram (e.g. ‘you’) appears in the text will often be much greater than the number of times a bigram or trigram (e.g. ‘you have’ or ‘you have to’) appears in the text).

Other features used by the system may include parts of speech tagging. For example, the system may provide more relevant information about the emotion content presented in the text that can be used. For instance, certain parts of speech may be more or less associated with specific emotions over others. Similar to word n-grams, POS features can also take the form of unigrams, bigrams or trigrams. Additionally or alternatively, the system may use other linguistic input that can be automatically generated by applying a natural language parser to an input text (e.g., grammatical constructions). Multiple different features can also be used simultaneously to train a (feature-based) model (such as SVMs).

Additionally or alternatively, the system may use word lemmas, which are important as in English, words can appear in inflected forms. In predicting emotion, the system may use semantic rather than the specific form that a word appears in. For example, if we encounter the words ‘walk’, ‘walked’, ‘walks’, or ‘walking’, the system may replace all of these with the base form ‘walk’.

The system may use vectorization (and/or other embedding) for generating a feature input based on the user actions and/or detected features. For example, the extracted text features may be converted into mathematical vectors in order to be understood as input by the multi-tier machine learning model. In one example, the system may use Term Frequency-Inverse Document Frequency vectorization (TF-IDF), where the number of times each word in the vocabulary occurs within and between examples is used to calculate an importance weight of that word in the example. Common words such as pronouns will occur frequently within individual text examples, but also in a high proportion of examples in the dataset, thus receiving a low importance weight for determining emotion labels. Words that occur frequently within a specific text example, but rarely in other text examples will receive the highest importance weights for determining that example's emotion labels.

FIG. 3C is an illustrative diagram of emotional state probability scores generated by a machine learning model of the multi-tier machine learning models, in accordance with one or more embodiments. For example, the system may output a probability (e.g., 0-1) for each of emotional state as shown in the diagram 350 (e.g., showing a subset of emotions). The tables show accuracy in the form of true positives (e.g., the correct label is used and the model predicts this), false positives, false negatives, and true negatives.

FIG. 3D is an illustrative diagram of the precision of the multi-tier machine learning models, in accordance with one or more embodiments. For example, diagram 370 illustrates the F1 scores of the model using different base models (e.g., a neural network model (DistilBert) and a Support Vector Machine (SVM)).

For example, the F-score or F-measure is a measure of a test's accuracy. It is calculated from the precision and recall of the test, where the precision is the number of true positive results divided by the number of all positive results, including those not identified correctly, and the recall is the number of true positive results divided by the number of all samples that should have been identified as positive. Precision is also known as positive predictive value, and recall is also known as sensitivity in diagnostic binary classification. The F1 score is the harmonic mean of the precision and recall.

It should be noted that the macro F1 score was 0.51 for the multi-tier machine learning model based on the support vector machine (“SVM”) classifier and 0.57 for the multi-tier machine learning model based on DistilBert model. The figures below indicate the percentage of true positives (model: 1, true: 1), false positives (model: 1, true: 0), true negatives (model 0, true: 0), and false negatives (model: 0, true: 1) when evaluating the performance of a trained neural network classifier. Diagram 370 also includes F1 scores for a range of emotions. For example, the neural network model had a macro F1 of roughly 0.6, and the SVM model had roughly 0.5.

FIG. 4 is an illustrative model architecture of a multi-tier machine learning model, in accordance with one or more embodiments. One tier of the multi-tiered machine learning model may include an artificial neural network (e.g., model 430) and another tier may include a SVM machine classifier (e.g., model 420). In some embodiments, a first machine learning model (e.g., model 420) is a supervised machine learning model and a second machine learning model (e.g., model 430) is an unsupervised machine learning model. It should be noted that alternatively, the first machine learning model (e.g., model 420) may be either a supervised or unsupervised machine learning model and/or the second machine learning model (e.g., model 430) may be a supervised or unsupervised machine learning model.

For example, model 430 (and/or other models described herein such as model 432 and model 434) may include neural networks that consist of a number of layers of nodes, connected by weights. During training, these weights are adjusted in order to optimise the predictions that are made at the output layer (e.g., output 440) of the networks.

In some embodiments, model 400 may predict an emotional state of a user. This emotional state may be selected from a plurality of emotional states stored by the system. Model 400 may first determine a context and then select an emotional state from the context. In some embodiments, the system may determine the emotional state from the plurality of emotional states based on the similar feature inputs. For example, the system may determine emotional states based on similar characteristics of the users. For example, the system may determine that users who ask different questions about friends have similar emotional states.

A multi-tiered approach may be used to capture this behaviour. The first layer of the model (e.g., model 420) identifies which emotional state is most likely, then in the subsequent layer, the model (e.g., model 430) identifies which emotional states are most likely.

In some embodiments, the model (e.g., model 400) may automatically perform actions based on output 440. In some embodiments, the model (e.g., model 400) may not perform any actions, rather the output of the model (e.g., model 400) may be only used to decide which mental health disorder recommendations to display to a user.

In some embodiments, one or more models included in model 400 may include a DistilBert model. This model architecture has been pre-trained on two tasks—masked language modelling, and next sentence prediction, providing the base model (e.g., model 420) with an understanding of language and context. The key differences of this model to the SVMSVM classifiers include the use of an embedding layer that transforms the input words into vectors, such that words that are closer in meaning are closer to each other in vector space (adding some semantic understanding). It should be noted that a difference between Distilbert and SVMs is that in the former the system may use a deep neural network that automatically identifies features at different levels of abstraction, whereas SVMs are feature-based machine learning models where the system may need input to determine what features should be used to train it.

Furthermore, one or more models included in model 400 (e.g., model 420) may incorporate attention mechanisms that allow it to understand context and how different words interact with each other. For example, the attention mechanisms may provide a better understanding of which nouns certain pronouns are related to (e.g. ‘it is’). This base model, with an understanding of natural language may then be easily fine-tuned for the specific task of emotion classification. For example, the system may include a task-specific output layer (e.g., output layer 436) such that it may make a prediction for how likely the input text is to express each emotion. Thus, the output of the model is a probability (0-1) for each of the 11 emotions.

For example, in the DistilBert model, text samples are processed in batches of 32 and are either padded or truncated to a length of 128. It has a hidden size of 768, and dropout was set to 0.3. DistilBert models are trained using the AdamW optimiser, with a fixed learning rate of 5e⁻⁵ for at least 5 epochs and until convergence is reached. Early stopping may be applied by the system by monitoring performance on the validation dataset.

Additionally or alternatively, model 420 may be structured as a SVM classifier. Model 420 may be a non-linear model and/or supervised learning model that can perform both classification and regression. Model 420 may perform these tasks by measuring interactions between variables within large datasets. In some embodiments, model 420 may be used to determine contexts for a feature input (e.g., feature input 410). For example, model 420 may be a general-purpose supervised learning algorithm that the system uses for both classification and regression tasks. It may be an extension of a linear model that is designed to capture interactions between features within high dimensional sparse datasets economically. For example, SVM classifiers are extensions of linear models which model the interactions of variables. They map and plot their interactions to a lower dimension. As a result, the number of parameters extends linearly through the dimensions.

In some embodiments, the feature input may include a vector that describes various information about a user, a user action (which may include user inactions), and/or a current or previous interaction with the user. The system may further select the information for inclusion in the feature input based on a predictive value. The information may be collected actively or passively by the system and compiled into a user profile.

In some embodiments, the information (e.g., a user action) may include conversation details such as information about a current session, including a channel or platform, e.g. desktop web, iOS, mobile, a launch page (e.g., the webpage that the application was launched from), a time of launch, and/or activities in a current or previous session before launching the application. The system may store this information and all the data about a conversational interaction may be available in real-time via HTTP messages and/or through data streaming from one or more sources (e.g., via an API.).

In some embodiments, the information (e.g., a user action) may include insights about users (e.g., related to mental health disorders), provided to the application (e.g., via an API) from one or more sources such as qualitative or quantitative representations (e.g., a percent) of a given activity (e.g., medical tests) in a given time period (e.g., six months), upcoming action, information from third parties, etc.

Model 420 may include embedding layers 424 at which each feature of the vector of feature input 410 is converted into a dense vector representation. These dense vector representations for each feature are then pooled to convert the set of embedding vectors into a single vector. The created vector is then used as an input for model 430. The output from the first machine learning model may then be input into a second machine learning model (or second tier). For example, the output may comprise the feature input, a determination of a context, and/or a specific model (or algorithm) for use in the second tier.

Model 430 may be structured as an artificial neural network (or a series of artificial neural networks). For example, model 430 may include model 432 and model 434. Each model within model 430 may include one or more hidden layers. Model 430 may be based on a large collection of neural units (or artificial neurons). Model 430 loosely mimics the manner in which a biological brain works (e.g., via large clusters of biological neurons connected by axons). Each neural unit of a model 430 may be connected with many other neural units of model 430. Such connections can be enforcing or inhibitory in their effect on the activation state of connected neural units. In some embodiments, each individual neural unit may have a summation function which combines the values of all of its inputs together. In some embodiments, each connection (or the neural unit itself) may have a threshold function such that the signal must surpass before it propagates to other neural units. Model 430 may be self-learning and trained, rather than explicitly programmed, and can perform significantly better in certain areas of problem solving, as compared to traditional computer programs.

Model 432 and model 434 may represent models based on different emotional frameworks. For example, model 432 may be based on 11 emotional states, whereas model 434 may be based on a Valence-Arousal-Dominance framework. For example, model 434 may use a dataset that contains 8,062 examples of text taken from a variety of sources such as newspapers, blogs and letters. Each text example has been pre-annotated with emotion labels using the aforementioned Valence-Arousal-Dominance framework, with each excerpt receiving a score from 1-5 on each of these dimensions. The dimensions are described as follows: valence—positive vs negative; arousal—calm vs excited; and dominance—being controlled vs being in control. The base neural network model (e.g., model 420) is the same DistilBert architecture mentioned above. However, when using the dimensional emotion framework, model 400 includes model 434 that outputs 3 scores (1-5) one for each dimension: valence, arousal, and dominance. In some embodiments, the dimensional framework warrants a different performance metric, as the system is now assessing predictions of a continuous measure. The system may evaluate performance using the root mean square error, which was 0.027 during testing.

For example, the system may use an encoder described above, though there are now two separate prediction heads for each type of emotional framework being predicted. This model therefore benefits from having a shared component with parameters that are trained jointly on both frameworks, and separate components with parameters that are specific to each task as well. This often improves performance relative to training on a single task alone, and aids in generalisation.

During training, output 440 may correspond to a classification of model 430 (e.g., an emotional state) and an input known to correspond to that classif1cation may be input into model 430 from model 420. In some embodiments, model 430 may include multiple layers (e.g., where a signal path traverses from front layers to back layers). In some embodiments, back propagation techniques may be utilized by model 430 where forward stimulation is used to reset weights on the “front” neural units. In some embodiments, stimulation and inhibition for model 430 may be more free-flowing, with connections interacting in a more chaotic and complex fashion. During testing, output 440 may indicate whether or not a given input corresponds to a classification of model 430 (e.g., whether or not a given output of model 420 corresponds to an emotional state).

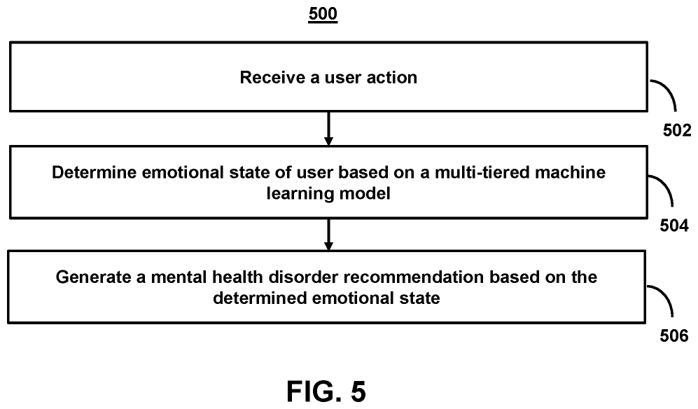

FIG. 5 shows a flowchart of the steps involved in generating mental health disorder recommendations using multi-tier machine learning models, in accordance with one or more embodiments. For example, process 500 may represent the steps taken by one or more devices as shown in FIGS. 1-2 when generating mental health disorder recommendations using multi-tier machine learning models.

At step 502, process 500 (e.g., using one or more components in system 200 (FIG. 2)) receives a user action. For example, the system may receive one or more user inputs to a user interface (e.g., user interface 100 (FIG. 1)). The system may then determine a likely emotional state of the user in order to generate one or more mental health disorder recommendations based on that emotional state. The user action may take various forms including speech commands, textual inputs, responses to system queries, and/or other user actions (e.g., logging into a mobile application of the system). In each case, the system may aggregate information about the user action, information about the user, and/or other circumstances related to the user action (e.g., time of day, previous user actions, current account settings, etc.) in order to determine a likely emotional state of the user.

At step 504, process 500 (e.g., using one or more components in system 200 (FIG. 2)) determines an emotional state of a user based on a multi-tier machine learning model. For example, the system may first use a first tier of a model (e.g., model 420 (FIG. 4)) to determine a context of the user's input. The system may then determine a second tier of a model (e.g., model 430 (FIG. 4)) to determine an emotional state of the user's emotional state. For example, the first machine learning model (or first tier) may be selected based on its attributes to generate results with sparse amounts of training data and/or in a supervised manner.

For example, the system may select a model for the second tier of the model (e.g., model 432 or model 434 (FIG. 4)) based on the context. For example, in some embodiments, the emotional state may be predicted with more reliability using one model over another. Additionally or alternatively, the system may use models that are trained jointly using both datasets mentioned above.

For example, the system may use an encoder described above, though there are now two separate prediction heads for each type of emotion framework being predicted. This model therefore benefits from having a shared component with parameters that are trained jointly on both frameworks, and separate components with parameters that are specific to each task as well. This often improves performance relative to training on a single task alone, and aids in generalisation.

At step 506, process 500 (e.g., using one or more components in system 200 (FIG. 2)) generates a mental health disorder recommendation based on the determined emotional state. For example, by using the multi-tier machine learning model, the system may ensure that a mental health disorder recommendation is generated based on an emotional state in the correct cluster. The system may also increase the likelihood that it determines a correct emotional state of the user. For example, as the initial determination of the context has been made (e.g., using a first machine learning model), the second machine learning model may be trained to optimize the precision of the selection of the emotional state. That is, the output of the second machine learning model, and the recommendation generated based on that output.

It is contemplated that the steps or descriptions of FIG. 5 may be used with any other embodiment of this disclosure. In addition, the steps and descriptions described in relation to FIG. 5 may be done in alternative orders or in parallel to further the purposes of this disclosure. For example, each of these steps may be performed in any order, in parallel, or simultaneously to reduce lag or increase the speed of the system or method. Furthermore, it should be noted that any of the devices or equipment discussed in relation to FIGS. 1-2 could be used to perform one or more of the steps in FIG. 5.

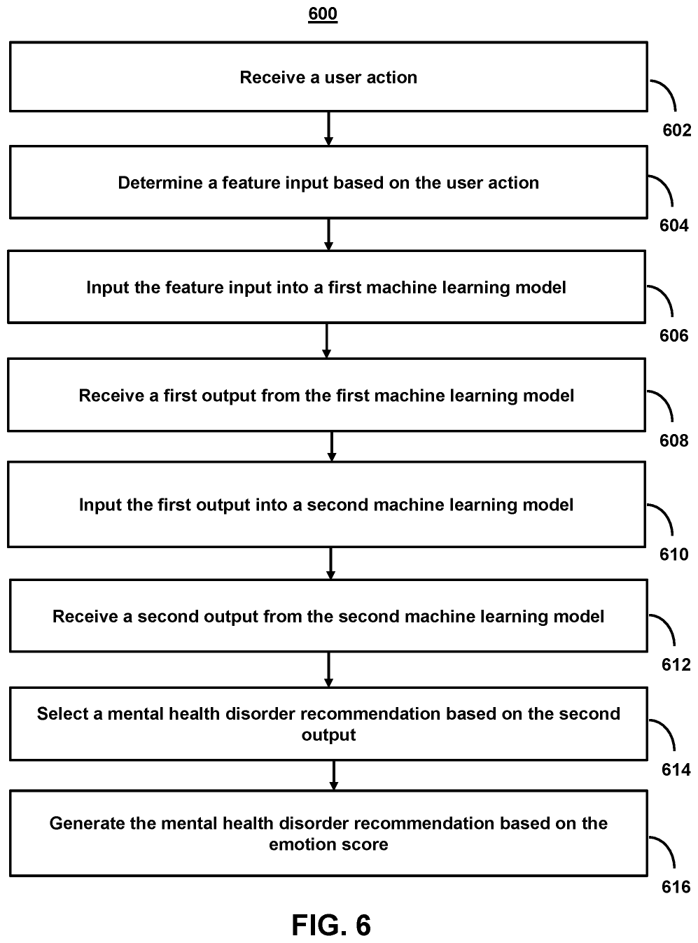

FIG. 6 shows a flowchart of the steps involved in generating mental health disorder recommendations using a two-tier machine learning model, in accordance with one or more embodiments. For example, process 600 may represent the steps taken by one or more devices as shown in FIGS. 1-2 when in generating mental health disorder recommendations.

At step 602, process 600 (e.g., using one or more components in system 200 (FIG. 2)) receives a user action. For example, the system may receive a first user action during a conversational interaction with a user interface as shown in FIG. 1.

At step 604, process 600 (e.g., using one or more components in system 200 (FIG. 2)) determines a feature input based on the user action. For example, the system may determine a first feature input based on the first user action in response to receiving the first user action. The system may generate the feature input based on one or more criteria. For example, the system may generate the feature input based on a part-of-speech tagging.

At step 606, process 600 (e.g., using one or more components in system 200 (FIG. 2)) inputs the feature input into a first machine learning model. For example, the system may input the first feature input into a first machine learning model, wherein the first machine learning model is trained to select a context from a plurality of contexts based on the first feature input, wherein each context of the plurality of contexts corresponds to a respective emotional state of a user.

In some embodiments, the system may receive a first labeled feature input, wherein the first labeled feature input is labeled with a known context for the first labeled feature input. The system may then train the first machine learning model to classify the first labeled feature input with the known context.

In some embodiments, the system may cluster available emotional states into one or more plurality of contexts. For example, the system may group and/or categorize emotional states into contexts based on similarities between the emotional states and/or similarities between the feature inputs. For example, two user actions that may appear similar may first be stored into the same context and then further classified into emotional states. This ensures that the system determines emotional states with an increased accuracy.

At step 608, process 600 (e.g., using one or more components in system 200 (FIG. 2)) receives a first output from the first machine learning model. For example, the system may receive a first output from the first machine learning model.

At step 610, process 600 (e.g., using one or more components in system 200 (FIG. 2)) inputs the first output into a second machine learning model. For example, the system may input the first output into a second machine learning model, wherein the second machine learning model is trained to select an emotional state from a plurality of emotional states of the selected context based on the first output, and wherein each emotional state of the plurality of emotional states corresponds to a respective emotional state of the user. In some embodiments, the second machine learning model may be an unsupervised machine learning model and/ or an artificial neural network model.

In some embodiments, the system may select the second machine learning model, from a plurality of machine learning models, based on the context selected from the plurality of contexts, wherein each context of the plurality of contexts corresponds to a respective machine learning model from the plurality of machine learning models. For example, the system may develop independent models, using different algorithms and/ or trained on different data, in order to increase the precision at which an emotional state is determined.

For example, the system may receive a second user action during the conversational interaction with the user interface. The system may determine a second feature input for the first machine learning model based on the second user action in response to receiving the second user action. The system may input the second feature input into the first machine learning model. The system may receive a different output from the first machine learning model, wherein the different output corresponds to a different context from the plurality of contexts. The system may input the different output into the second machine learning model.

At step 612, process 600 (e.g., using one or more components in system 200 (FIG. 2)) receives a second output from the second machine learning model. For example, the system may receive, using the control circuitry, a second output from the second machine learning model. In some embodiments, the system may receive a first labeled output from the first machine learning model, wherein the first labeled output is labeled with a known emotional state. The system may then train the second machine learning model to classify the first labeled output with the known emotional state.

At step 614, process 600 (e.g., using one or more components in system 200 (FIG. 2)) selects a mental health disorder recommendation based on the second output. For example, the system may select, using the control circuitry, a mental health disorder recommendation from a plurality of mental health disorder recommendations based on the second output. For example, the system may have one or more potential responses and select one or more of these responses based on the predicted emotional state of the user.

At step 616, process 600 (e.g., using one or more components in system 200 (FIG. 2)) generates the mental health disorder recommendation. For example, the system may generate, at a user interface, the mental health disorder recommendation following the conversational interaction (e.g., as shown in FIG. 1).

It is contemplated that the steps or descriptions of FIG. 6 may be used with any other embodiment of this disclosure. In addition, the steps and descriptions described in relation to FIG. 6 may be done in alternative orders or in parallel to further the purposes of this disclosure. For example, each of these steps may be performed in any order, in parallel, or simultaneously to reduce lag or increase the speed of the system or method. Furthermore, it should be noted that any of the devices or equipment discussed in relation to FIGS. 1-2 could be used to perform one or more of the steps in FIG. 6.

The above-described embodiments of the present disclosure are presented for purposes of illustration and not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

The present techniques will be better understood with reference to the following enumerated embodiments:

1. A method for generating mental health disorder recommendations using multi-tier machine learning models, the method comprising: receiving a first user action during a conversational interaction with a user interface; in response to receiving the first user action, determining a first feature input based on the first user action; inputting the first feature input into a first machine learning model, wherein the first machine learning model is trained to select a context from a plurality of contexts based on the first feature input, wherein each context of the plurality of contexts corresponds to a respective emotional state of a user, receiving a first output from the first machine learning model, inputting the first output into a second machine learning model, wherein the second machine learning model is trained to select an emotional state from a plurality of emotional states of the selected context based on the first output, and wherein each emotional state of the plurality of emotional states corresponds to a respective emotional state of the user, receiving a second output from the second machine learning model, selecting a mental health disorder recommendation from a plurality of mental health disorder recommendations based on the second output, and generating the mental health disorder recommendation following the conversational interaction.

2. The method of embodiment 2, further comprising selecting the second machine learning model, from a plurality of machine learning models, based on the context selected from the plurality of contexts, wherein each context of the plurality of contexts corresponds to a respective machine learning model from the plurality of machine learning models.

3. The method of any one of embodiments 1-2, further comprising: receiving a second user action during the conversational interaction with the user interface, in response to receiving the second user action, determining a second feature input for the first machine learning model based on the second user action, inputting the second feature input into the first machine learning model, receiving a different output from the first machine learning model, wherein the different output corresponds to a different context from the plurality of contexts, and inputting the different output into the second machine learning model.

4. The method of any one of embodiments 1-3, wherein the first machine learning model is a supervised machine learning model, and wherein the second machine learning model is a supervised machine learning model.

5. The method of any one of embodiments 1-4; wherein the first machine learning model is a support vector machine classifier, and wherein the second machine learning model is an artificial neural network model.

6. The method of any one of embodiments 1-5; further comprising clustering available emotional states into the plurality of contexts.

7. The method of any one of embodiments 1-6; further comprising: receiving a first labelled feature input, wherein the first labelled feature input is labelled with a known context for the first labelled feature input; and training the first machine learning model to classify the first labelled feature input with the known context.

8. The method of any one of embodiments l-7; wherein the first feature input is a vectorization of a conversational detail or information from a user account of the user.

9. The method of any one of embodiments 1-8; wherein the first feature input indicates a vectorization of an n-grams corresponding to the first user action.

10. The method of any one of embodiments 1-9; wherein the first feature input indicates a vectorization of a part-of-speech corresponding to the first user action.

11. The method of any one of embodiments 1-9; further comprising: determining user information corresponding to the mental health disorder recommendation; determining a network location of the user information; and generating a network pathway to the user information.

12. The method of embodiment 10; further comprising: automatically retrieving the user information from the network location based the mental health disorder recommendation; and generating for display the user information on a second user interface.

13. A tangible; non-transitory; machine-readable medium storing instructions that; when executed by a data processing apparatus; cause the data processing apparatus to perform operations comprising those of any of embodiments 1-12.

14. A system comprising: one or more processors; and memory storing instructions that; when executed by the processors; cause the processors to effectuate operations comprising those of any of embodiments 1-12.

15. A system comprising means for performing any of embodiments 1-12.

1. A system for generating mental health disorder recommendations using multi-tier machine learning models, the system comprising:
cloud-based storage circuitry configured to:
store a first machine learning model, wherein the first machine learning model is trained to select a context from a plurality of contexts based on user actions, and wherein each context of the plurality of contexts corresponds to a respective emotional state of a user following a first user action of a text input; and
store a second machine learning model, wherein the second machine learning model is trained to select an emotional state from a plurality of emotional states of a selected context based on a first output, and wherein each emotional state of the plurality of emotional states corresponds to a respective emotional state of the user;
cloud-based control circuitry configured to:
receive the first user action during a conversational interaction with a first user interface;
determine a first feature input based on the first user action in response to receiving the first user action, wherein the first feature input is a vectorization that calculates an importance weight of each word of a conversational detail or information from a user account of the user;
input the first feature input into the first machine learning model;
receive the first output from the first machine learning model by selecting the context from the first feature input that most clearly depicts the context of the user, the first output indicative of a selected context of the plurality of contexts;
select the second machine learning model, from a plurality of machine learning models, based on the selected context, wherein each context of the plurality of contexts corresponds to a respective machine learning model from the plurality of machine learning models;
input the first output in response to receiving, using the control circuitry, a second output from the second machine learning model, the second output being the emotional state determined from the context of the first output that most closely depicts the emotional state of the user; and
select a mental health disorder recommendation from a plurality of mental health disorder recommendations based on the second output; and
cloud-based input/output circuitry configured to:
transmit, to a second user interface, the mental health disorder recommendation following the conversational interaction.

2. A method for generating mental health disorder recommendations using multi-tier machine learning models, the method comprising:
receiving a first user action of a text input during a conversational interaction with a user interface;
in response to receiving the first user action, determining, using control circuitry, a first feature input based on the first user action, wherein the first feature input is a vectorization that calculates an importance weight of each word of a conversational detail or information from a user account of the user;
inputting, using the control circuitry, the first feature input into a first machine learning model, wherein the first machine learning model is trained to select a context from a plurality of contexts based on user actions, and wherein each context of the plurality of contexts corresponds to a respective emotional state of a user;
receiving, using the control circuitry, a first output from the first machine learning model by selecting the context from the first feature input that most clearly depicts the context of the user;
inputting, using the control circuitry, the first output into a second machine learning model, wherein the second machine learning model is trained to select an emotional state from a plurality of emotional states of the selected context based on the first output, and wherein each emotional state of the plurality of emotional states corresponds to a respective emotional state of the user;
receiving, using the control circuitry, a second output from the second machine learning model, the second output being the emotional state determined from the context of the first output that most closely depicts the emotional state of the user;
selecting, using the control circuitry, a mental health disorder recommendation from a plurality of mental health disorder recommendations based on the second output; and
generating the mental health disorder recommendation following the conversational interaction.

3. The method of claim 2, further comprising of selecting the second machine learning model, from a plurality of machine learning models, based on the context selected from the plurality of contexts, wherein each context of the plurality of contexts corresponds to a respective machine learning model from the plurality of machine learning models.

4. The method of claim 2, further comprising:
receiving a second user action during the conversational interaction with the user interface;
in response to receiving the second user action, determining a second feature input for the first machine learning model based on the second user action;
inputting the second feature input into the first machine learning model;
receiving a different output from the first machine learning model, wherein the different output corresponds to a different context from the plurality of contexts; and
inputting the different output into the second machine learning model.

5. The method of claim 2, wherein the first machine learning model is a supervised machine learning model, and wherein the second machine learning model is a supervised machine learning model.

6. The method of claim 2, wherein the first machine learning model is a support vector machine classifier, and wherein the second machine learning model is an artificial neural network model.

7. The method of claim 2, The method of claim 2, wherein the first feature input is a vectorization of a conversational detail or information from a user account of the user.

8. The method of claim 2, further comprising:
receiving a first labelled feature input, wherein the first labelled feature input is labelled with a known context for the first labelled feature input; and
training the first machine learning model to classify the first labelled feature input with the known context.

9. The method of claim 2, wherein the first feature input is a vectorization of an n- grams corresponding to the first user action, a vectorization of a part-of-speech corresponding to the first user action.

10. The method of claim 2, further comprising:
determining user information corresponding to the mental health disorder recommendation;
determining a network location of the user information; and
generating a network pathway to the user information.

11. The method of claim 10, further comprising:
automatically retrieving the user information from the network location based the mental health disorder recommendation; and
generating for display the user information on a second user interface.

12. A non-transitory computer-readable medium for generating mental health disorder recommendations using multi-tier machine learning models, comprising of instructions that, when executed by one or more processors, cause operations comprising:
receiving a first user action of a text input during a conversational interaction with a user interface;
in response to receiving the first user action, determining a first feature input based on the first user action, wherein the first feature input is a vectorization that calculates an importance weight of each word of a conversational detail or information from a user account of the user;
inputting the first feature input into a first machine learning model, wherein the first machine learning model is trained to select a context from a plurality of contexts based on user actions, and wherein each context of the plurality of contexts corresponds to a respective emotional state of a user;
receiving a first output from the first machine learning model by selecting the context from the first feature input that most clearly depicts the context of the user;
inputting the first output into a second machine learning model, wherein the second machine learning model is trained to select an emotional state from a plurality of emotional states of the selected context based on the first output, and wherein each emotional state of the plurality of emotional states corresponds to a respective emotional state of the user;
receiving a second output from the second machine learning model, the second output being the emotional state determined from the context of the first output that most closely depicts the emotional state of the user;
selecting a mental health disorder recommendation from a plurality of mental health disorder recommendations based on the second output; and
generating the mental health disorder recommendation following the conversational interaction.

13. The non-transitory computer-readable medium of claim 12, further comprising of instructions that cause further operations comprising of selecting the second machine learning model, from a plurality of machine learning models, based on the context selected from the plurality of contexts, wherein each context of the plurality of contexts corresponds to a respective machine learning model from the plurality of machine leaming models.

14. The non-transitory computer-readable medium of claim 12, further comprising of instructions that cause further operations comprising:
receiving a second user action during the conversational interaction with the user interface;
in response to receiving the second user action, determining a second feature input for the first machine learning model based on the second user action;
inputting the second feature input into the first machine learning model;
receiving a different output from the first machine learning model, wherein the different output corresponds to a different context from the plurality of contexts; and
inputting the different output into the second machine learning model.

15. The non-transitory computer-readable medium of claim 12, wherein the first machine learning model is a supervised machine learning model, and wherein the second machine learning model is a supervised machine learning model.

16. The non-transitory computer-readable medium of claim 12, wherein the first machine learning model is a support vector machine classifier, and wherein the second machine learning model is an artificial neural network model.

17. The non-transitory computer-readable medium of claim 12, The method of claim 2, wherein the first feature input is a vectorization of a conversational detail or information from a user account of the user.

18. The non-transitory computer-readable medium of claim 12, further comprising of instructions that cause further operations comprising:
receiving a first labelled feature input, wherein the first labelled feature input is labelled with a known context for the first labelled feature input; and
training the first machine learning model to classify the first labelled feature input with the known context.

19. The non-transitory computer-readable medium of claim 12, wherein the first feature input includes a vectorization of an n-grams corresponding to the first user action, or a vectorization of a part-of-speech corresponding to the first user action.

20. The method of claim 2, further comprising:
determining user information corresponding to the mental health disorder recommendation;
determining a network location of the user information;
generating a network pathway to the user information;
automatically retrieving the user information from the network location based the mental health disorder recommendation; and
generating for display the user information on a second user interface.
